(19) United States
(12) Patent Application Publication
(10) Pub. No.: US 2003/0049616 A1
(43) Pub. Date: Mar. 13, 2003

(54) Enzymatic synthesis of oligonucleotide tags

(76) Inventors: Sydney Brenner, La Jolla, CA (US); Steven R. Williams, San Francisco, CA (US)

Correspondence Address: PERKINS COIE LLP, P.O. BOX 2168, MENLO PARK, CA, 94026, US

(21) Appl. No.: 09/756,830

(22) Filed: Jan. 8, 2001

(51) Int. Cl. C12Q 1/68; C07H 21/04; C12P 19/34

(52) U.S. Cl. 435/6; 435/91.2; 435/320.1; 536/23.1

(57) ABSTRACT

The invention provides oligonucleotide tag compositions and methods for synthesizing repertoires of error-free oligonucleotide tags that may be used for labeling and sorting polynucleotides, such as cDNAs, restriction fragments, and the like. In accordance with the method of the invention, oligonucleotide tag precursors are provided in an amplicon, wherein the tag precursors each consists of one or more oligonucleotide “words” selected from the same minimally cross-hybridizing set of words. The oligonucleotide tag precursors are elongated by repeated cycles of cleavage, ligation of one or more words, and amplification. Cycles continue until the oligonucleotide tags of the repertoire have a desired length or complexity.

### FIELD OF THE INVENTION

The invention relates generally to methods for synthesizing collections of minimally cross-hybridizing oligonucleotide tags for identifying, sorting, and/or tracking molecules, especially polynucleotides.

### BACKGROUND

Specific hybridization of oligonucleotides and their analogs is a fundamental process that is employed in a wide variety of research, medical, and industrial applications, including the identification of disease-related polynucleotides in diagnostic assays, screening for clones of novel target polynucleotides, identification of specific polynucleotides in blots of mixtures of polynucleotides, amplification of specific target polynucleotides, therapeutic blocking of inappropriately expressed genes, DNA sequencing, and the like, e.g. Sambrook et al, Molecular Cloning: A Laboratory Manual, 2nd Edition (Cold Spring Harbor Laboratory, New York, 1989); Keller and Manak, DNA Probes, 2nd Edition (Stockton Press, New York, 1993); Milligan et al, J. Med Chem., 36: 1923-1937 (1993); Drmanac et al, Science, 260: 1649-1652 (1993); Bains, J. DNA Sequencing and Mapping, 4: 143-150 (1993).

Specific hybridization has also been proposed as a method of tracking, retrieving, and identifying compounds labeled with oligonucleotide tags, e.g. Brenner, International application PCT/US95/12791: Church et al, Science, 240: 185-188 (1988); Brenner and Lerner, Proc. Natl. Acad. Sci., 89: 5381-5383 (1992); Alper, Science, 264: 1399-1401 (1994); Cheverin et al, Biotechnology, 12. 1093-1099 (1994); and Needels et al, Proc Natl. Acad. Sci., 90: 10700-10704 (1993). The successful implementation of such tagging and sorting schemes depends in large part on the success in achieving specific hybridization between a tag and its complement. That is, for an oligonucleotide tag to successfully identify a substance, the number of false positive and false negative signals brought about by incorrect hybridizations must be minimized. And for oligonucleotide tags to effectively sort molecules, the number of tags hybridized to complements at incorrect sites must be minimized. Unfortunately. incorrect hybridizations brought about by the creation of stable duplexes containing mismatches are not uncommon because base pairing and base stacking free energies vary widely among nucleotides in a duplex or triplex structure. For example, a duplex consisting of a repeated sequence of deoxyadenosine (A) and thymidine (T) bound to its complement may have less stability than an equal-length duplex consisting of a repeated sequence of deoxyguanosine (G) and deoxycytidine (C) bound to a partially complementary target containing a mismatch. Thus, if a desired compound from a large combinatorial chemical library were tagged with the former oligonucleotide, a significant possibility would exist that, under hybridization conditions designed to detect perfectly matched AT-rich duplexes, undesired compounds labeled with the GC-rich oligonucleotide—even in a mismatched duplex—would be detected or sorted along with the perfectly matched duplexes consisting of the AT-rich tag. Even though reagents, such as tetramethylammonium chloride, are available to negate base-specific stability differences of oligonucleotide duplexes, the effect of such reagents is often limited and their presence can be incompatible with, or render more difficult, further manipulations of the selected compounds, e.g. amplification by polymerase chain reaction (PCR), or the like.

Such problems have been addressed in the “solid phase” cloning technique, described in Brenner, International application PCT/US95/1279 1, by the development of oligonucleotide tags synthesized combinatorially from a set of so-called minimally cross-hybridizing oligonucleotides, or “words.” The words, which are oligonucleotides usually 3 to 6 nucleotides in length, differ from every other member of the same set by at least two nucleotides. Thus, a given word cannot form a duplex with the complement of any other word of the set without less than two mismatches. Of course, minimally cross-hybridizing sets are preferably formed from words differing from one another by even more than two nucleotides.

In such a scheme, different oligonucleotide tags constructed from concatenations of such words will differ from one another by at least two nucleotides, or by at least the number of nucleotides that their component words differ by. Therefore, by judiciously selecting word length, differences between words in a set, and the number of words per tag, one can obtain a large set, or repertoire, of oligonucleotide tags that each differ from one another by a significant percentage of their nucleotides. Such repertoires permit tagging and sorting of molecules with a much higher degree of specificity than ordinary oligonucleotides.

Unfortunately, current methods of solid phase synthesis, although highly efficient, still lead to a significant fraction of failure sequences when oligonucleotide tags start to exceed 30 to 40 nucleotides in length. The presence of such failure sequences can have a significant impact on solid phase cloning and sorting schemes, such as the one described in Brenner (cited above). When tag complements are synthesized separately from their corresponding oligonucleotide tags, the presence of different sets of failure sequences among the two reaction products means that not every oligonucleotide from one reaction will necessarily have a complementary oligonucleotide among products of the other reaction. In particular, failure sequences produced in one reaction will generally not have complementary failure sequences produced in the other reaction. While this is not a problem for tag complements combinatorially synthesized on solid phase supports because the number and kind of failures are randomly distributed among a population of predominantly correct-sequence oligonucleotides, for tags attached to DNAs which are sampled and amplified, a significant probability exists that if one or more of the sampled tags contain failure sequences, no solid phase supports will exist for them that has a population of perfect complements. Consequently, DNAs with such tags cannot be effectively sorted.

In view of the above, it would be useful if there were available a method of producing oligonucleotide tags which would avoid or minimize the chance of there being sampled and amplified tags that contain failure sequences.

### SUMMARY OF THE INVENTION

Accordingly, objectives of my invention include, but are not limited to, providing a method of synthesizing oligonucleotide tags which minimizes the production of failure sequences;

providing an enzymatic method of synthesizing oligonucleotide tags by the combinatorial addition of words; providing a method of convergent synthesis of oligonucleotide tags from error-free components; providing a method of constructing tag-DNA conjugates whose tags are free of failure sequences; providing compositions comprising novel oligonucleotide tags.

My invention achieves these and other objectives by providing a method of synthesizing oligonucleotide tags that comprises successive cycles of cleavage of a oligonucleotide tag precursor to permit the ligation of one or more words from a minimally cross-hybridizing set, ligation of the one or more words, and amplification of ligated structure. Preferably, repertoires of oligonucleotide tags of a predetermined length are assembled from words, or sub-assemblies of words, that are free of failure sequences. Preferably, such error-free words or sub-assemblies of words are obtained either by separately synthesizing and sequencing individual words or sub-assemblies of words prior to assembly, or by successive ligations of adaptors having protruding strands consisting of word sequences that select complementary word sequences on the protruding strand of a growing tag. Preferably, in the former embodiment, words or sub-assemblies of words are inserted into and maintained in conventional cloning vectors, after which they are sequenced to confirm that no errors are present. For use in the method of the invention, the words or sub-assemblies of words are excised from the vectors, mixed, and ligated to an oligonucleotide tag precursor. Preferably, in the latter embodiment, error-containing words are excluded from the assembly process by requiring that the single stranded form of each added word anneal to a perfectly matched complement of an oligonucleotide tag precursor in a ligation step. If a mismatch exists because a failure sequence is present in one of the strands, no ligation will take place, either precluding further growth of the tag if the failure is carried by its protruding strand, or promoting the annealing of a different word if the failure is carried by the word being added.

The invention further includes repertoires of oligonucleotide tags consisting of a plurality words wherein at least two words of the plurality are separated by one or two nucleotides.

The present invention overcomes difficulties in sorting polynucleotides with oligonucleotide tags synthesized by currently available methods. By providing oligonucleotide tags free of failure sequences, sampled and amplified tag-polynucleotide conjugates are assured of finding a tag complement with which to form a perfectly matched duplex.

### BRIEF DESCRIPTION OF THE DRAWINGS

FIG. 1a illustrates a preferred embodiment of the invention in which oligonucleotide tags are assembled by successive additions of one or more words to an oligonucleotide tag precursor.

FIG. 1b illustrates a preferred embodiment of the invention in which oligonucleotide tags are assembled by convergent additions of increasingly larger sub-assemblies of words.

FIG. 2 illustrates a preferred embodiment of the invention wherein oligonucleotide tags are assemble by successive additions and self-selection of words to an oligonucleotide tag precursor.

### DEFINITIONS

As used herein, the term “word” means an oligonucleotide selected from a minimally cross-hybridizing set of oligonucleotides, as disclosed in U.S. Pat. No. 5,604,097; International patent application PCT/US96/09513; and allowed U.S. patent application Ser. No. 08/659,453; which references are incorporated by reference. An oligonucleotide tag of the invention consists of a plurality of words, or oligonucleotide subunits, that are selected from the same minimally cross-hybridizing set. In such a set, a duplex or triplex consisting of a word of the set and the complement of any other word of the same set contains at least two mismatches. Preferably, a duplex or triplex consisting of a word of the set and the complement of any other word of the same set contains an even larger minimum number of mismatches, e.g 3, 4, 5, or 6, depending on the length of the words. Still more preferably, the minimum number of mismatches is either 1, 2, or 3 less than the length of the word. Most preferably, the minimum number of mismatches is 1 or 2 less than the length of the word.

“Complement” or “tag complement” as used herein in reference to oligonucleotide tags refers to an oligonucleotide to which a oligonucleotide tag specifically hybridizes to form a perfectly matched duplex or triplex. In embodiments where specific hybridization results in a triplex, the oligonucleotide tag may be selected to be either double stranded or single stranded. Thus, where triplexes are formed, the term “complement” is meant to encompass either a double stranded complement of a single stranded oligonucleotide tag or a single stranded complement of a double stranded oligonucleotide tag. Usually, populations of identical tag complements are attached to a spatially defined region of a solid phase support. Preferably, such solid phase supports are microparticles and the defined region is the entire surface of the microparticle.

The term “oligonucleotide” as used herein includes linear oligomers of natural or modified monomers or linkages, including deoxyribonucleosides, ribonucleosides, anomeric forms thereof, peptide nucleic acids (PNAs), and the like, capable of specifically binding to a target polynucleotide by way of a regular pattern of monomer-to-monomer interactions, such as Watson-Crick type of base pairing, base stacking, Hoogsteen or reverse Hoogsteen types of base pairing, or the like. Usually monomers are linked by phosphodiester bonds or analogs thereof to form oligonucleotides ranging in size from a few monomeric units, e.g 3-4, to several tens of monomeric units. Whenever an oligonucleotide is represented by a sequence of letters, such as “ATGCCTG,” it will be understood that the nucleotides are in 5′→3′ order from left to right and that upper or lower case “A” denotes deoxyadenosine, upper or lower case “C” denotes deoxycytidine, upper or lower case “G” denotes deoxyguanosine, and upper or lower case “T” denotes thymidine, unless otherwise noted. Analogs of phosphodiester linkages include phosphorothioate, phosphorodithioate, phosphoranilidate, phosphoramidate, and the like. Usually oligonucleotides of the invention comprise the four natural nucleotides; however, they may also comprise non-natural nucleotide analogs. It is clear to those skilled in the art when oligonucleotides having natural or non-natural nucleotides may be employed, e.g. where processing by enzymes is called for, usually oligonucleotides consisting of natural nucleotides are required.

“Perfectly matched” in reference to a duplex means that the poly- or oligonucleotide strands making up the duplex form a double stranded structure with one other such that every nucleotide in each strand undergoes Watson-Crick basepairing with a nucleotide in the other strand. The term also comprehends the pairing of nucleoside analogs, such as deoxyinosine, nucleosides with 2-aminopurine bases, and the like, that may be employed. In reference to a triplex, the term means that the triplex consists of a perfectly matched duplex and a third strand in which every nucleotide undergoes Hoogsteen or reverse Hoogsteen association with a basepair of the perfectly matched duplex. Conversely, a “mismatch” in a duplex between a tag and an oligonucleotide means that a pair or triplet of nucleotides in the duplex or triplex fails to undergo Watson-Crick and/or Hoogsteen and/or reverse Hoogsteen bonding.

As used herein, the term “complexity” in reference to a population of polynucleotides means the number of different species of molecule present in the population.

As used herein, the term “failure sequence” refers to a synthetic oligonucleotide or polynucleotide that does not have the correct, or intended, length and/or sequence because of a failure in a step of the synthetic process, e.g. spurious chain initiation, failure of a coupling step, failure of a capping step, chain scission, or the like.

As used herein, “amplicon ” means the product of an amplification reaction. That is, it is a population of polynucleotides, usually double stranded, that are replicated from a few starting sequences. Preferably, amplicons are produced either in a polymerase chain reaction (PCR) or by replication in a cloning vector.

### DETAILED OF THE INVENTION

The invention provides an enzymatic method for synthesizing a repertoire of oligonucleotide tags whose members are substantially free of failure sequences. Oligonucleotide tags are combinatorially synthesized by the assembly of error-free words or sub-assemblies of words in a series of enzymatic steps. Generally, the method of the invention comprises the following steps: (a) providing a repertoire of oligonucleotide tag precursors in an amplicon, the oligonucleotide tag precursors each comprising one or more words, and each of the one or more words being selected from the same minimally cross-hybridizing set; (b) cleaving the amplicon at a word in each of the oligonucleotide tag precursors to form one or more ligatable ends on each oligonucleotide tag precursor; (c) ligating one or more words to the one or more ligatable ends to elongate each of the oligonucleotide tag precursors; (d) amplifying the elongated oligonucleotide tag precursors in the amplicon; and (e) repeating steps (b) through (d) until a repertoire of oligonucleotide tags having the predetermined length is formed. The repertoire of oligonucleotide tags of the desired length contained in the final amplicon may then inserted into a convenient cloning vector, as taught by Brenner et al, International patent application PCT/US96/095 13. Preferably, each of the oligonucleotide tag precursors has the same length, which is determined by word length, the number of words making up the initial oligonucleotide tag precursor, and the stage of the assembly process, i.e. how many words or sub-assemblies of words have been added by operation of the method of the invention. Preferably, the amplicon of the method is a population of cloning vectors wherein different oligonucleotide tags or oligonucleotide tag precursors are represented in equal proportions as inserts of such vectors Preferably, whenever the oligonucleotide tag precursors are cleaved for the ligation of an additional word or sub-assembly of words, the cleavage takes place at the same word for all the oligonucleotide tag precursors of the repertoire. Preferably, the step of cleaving is carried out with a type IIs restriction endonuclease which cleaves at the same word for all the oligonucleotide tag precursors of the repertoire and produces ligatable ends having protruding strands. As used herein, the term “ligatable ends” means ends of a double stranded DNA that can be ligated to another double stranded DNA, including blunt-end ligation and “sticky” end ligation. Preferably, ligatable ends are sticky ends.

The invention further includes repertoires of oligonucleotide tags defined by the following formula.

w₁(N)ₓ₁w₂(N)ₓ₂ . . . (N)ₓₙ₋₁wₙ

wherein w₁, w₂, . . . w_{N} are words selected from the same minimally cross-hybridizing set, the words having a length of from three to fourteen nucleotides or basepairs; n is an integer in the range of from 4 to 10; N is a nucleotide or basepair; and x₁, x₂, . . . xₙ₋₁ are each an integer indicating how many nucleotides or basepairs, N, are present at the given location in the sequence of words, x₁, x₂, . . . Xₙ₋₁ each being selected from the group consisting of 0, 1, 2, 3, and 4, provided that at least one of x₁, x₂, . . . xₙ₋₁ is 1, 2, 3, or 4. Preferably, x₁, x₂, . . . xₙ₋₁ are each selected from the group consisting of 0, 1, and 2, provided that at least one of x₁, x₂, . . . xₙ₋₁ is 1 or 2. Preferably oligonucleotide tags of the above formula are synthesized by the method of the invention.

Preferably, words are from three to fourteen nucleotides or basepairs in length; and more preferably, words are from four to six nucleotides or basepairs in length. Most preferably, words are four nucleotides or basepairs in length. Usually, words consist of a linear sequence of nucleotides selected from the group consisting of A, C, G, and T. For words constructed from 3 of the 4 natural nucleotides, the following word sizes, differences between words of the same set, and set sizes are preferred.

| | Difference | |
|---|---|---|
| Word Length | Between Words | Set Size |
| 4 | 3 | 8 |
| 5 | 4 | 6 |
| 6 | 4 | 9 |
| 7 | 5 | 8 |
| 8 | 5 | 16 |
| 8 | 6 | 9 |

In some embodiments employing words of the above characteristics, subsets of the computed sets may be employed so that only words having specified GC content, melting temperature, reduced likelihood of self annealing, hairpin formation, or the like, are used to form tags. The above set sizes were computed using the algorithms listed in Brenner et al, PCT/US96/09513 and allowed U.S. patent application Ser. No. 08/659,453. Exemplary minimally cross-hybridizing sets of words for use with the invention are listed in the following table:

TABLE I
Exemplary Sets of Minimally Cross-Hybridizing
Words

| | Number of Nucleotides | | | |
|---|---|---|---|---|
| | per Word (Minimal No. | | | |
| | of Mismatches) | | | |
| 4(3) | 5(4) | 6(4) | 7(5) | 8(5) |
| gatt | tagta | gattag | gtaaaat | atgagtat |
| tgat | aaaag | agagtt | aaaagga | aggaagtg |
| taga | agggt | agttga | aaggaag | agggtaga |
| tttg | ggtaa | gagatt | aattttt | agttgaag |
| gtaa | gtatt | gttggt | ggaggtg | gagatggt |
| agta | tttgg | tggttg | gggtaga | gaggatag |
| atgt | | ttagag | tgtataa | gagtgata |
| aaag | | ttgaga | ttattgg | ggaagtga |
| | | atgtat | | ggatagat |
| | | | | gtaatatg |
| | | | | gttgggaa |
| | | | | tatagttg |
| | | | | tattagga |
| | | | | tgtgttat |
| | | | | ttatgagt |
| | | | | ttgttgag |

The length of oligonucleotide tags in a repertoire may vary widely depending on several factors, including the size or complexity of the repertoire desired, the difficulty in synthesizing corresponding tag complements on solid phase supports, the particular application, and the like. Generally, longer oligonucleotide tags permit the generation of larger repertoires; however, reliable synthesis of tag complements that exceed 40-50 nucleotides becomes increasingly difficult and monitoring and/or exercising quality control of mixtures of oligonucleotides becomes increasingly difficult as complexity increases. Thus, selection of particular tag lengths and complexities requires design tradeoffs by a practitioner of ordinary skill. Preferably, oligonucleotide tags of the invention are in the range of from 18 to 60 nucleotides in length. More preferably, oligonucleotide tags are in the range of from 18 to 40 nucleotides in length.

Preferably, minimally cross-hybridizing sets comprise words that make approximately equivalent contributions to duplex stability as every other word in the set. In this way, the stability of perfectly matched duplexes between every word and its complement is approximately equal. Guidance for selecting such sets is provided by published techniques for selecting optimal PCR primers and calculating duplex stabilities, e.g. Rychlik et al, Nucleic Acids Research, 17: 8543-855 (1989) and 18: 6409-6412 (1990); Breslauer et al, Proc. Natl. Acad. Sci., 83: 3746-3750 (1986); Wetmur, Crit. Rev. Biochem. Mol. Biol., 26: 227-259 (1991);and the like. For shorter tags, e.g. about 30 nucleotides or less, the algorithm described by Rychlik and Wetmur is preferred, and for longer tags, e.g. about 30-35 nucleotides or greater, an algorithm disclosed by Suggs et al, pages 683-693 in Brown, editor, ICN-UCLA Symp. Dev. Biol., Vol. 23 (Academic Press, New York, 1981) may be conveniently employed. Clearly, the are many approaches available to one skilled in the art for designing sets of minimally cross-hybridizing words within the scope of the invention. For example, to minimize the effects of different base-stacking energies of terminal nucleotides when words are assembled, words may be provided that have the same terminal nucleotides. In this way, when subunits are linked, the sum of the base-stacking energies of all the adjoining terminal nucleotides will be the same, thereby reducing or eliminating variability in tag melting temperatures.

For use with the invention, words or sub-assemblies of words are initially synthesized as single stranded oligonucleotides using conventional solid phase synthetic methods, e.g. using a commercial DNA synthesizer, such as PE Applied Biosystems (Foster City, Calif.) model 392 DNA synthesizer, or like instrument. Preferably, the words or sub-assemblies of words are synthesized within a longer oligonucleotide having appropriate restriction endonuclease recognition sites and primer binding sites to facilitate later manipulation. Preferably, such chemically synthesized oligonucleotides are rendered double stranded by providing a primer which binds to one end of the oligonucleotides and which is extended the length of the oligonucleotides with a DNA polymerase in the presence of the four dNTPs. For example, in a preferred embodiment the following oligonucleotide (shown in the 5′→3′ orientation) containing two words may be synthesized chemically (SEQ ID NO: 1):

```
                Pst I Bse RI Bbs I Bsp 120 Bbv I Hind III
                  ↓ ↓ ↓ ↓ ↓ ↓
cgacacctgcagaggagatgaagacga[word][word]gggcccatgctgcaagcttaccg
                                                          Formula I
```

In this example, forward and reverse primers shown below may be used to render the oligonucleotide double stranded so that the indicated restriction endonuclease recognition sites are formed.

| | |
|---|---|
| 5′-cgacacctgcagaggag | 5′-FAM-cggtaagcttgcagcat |
| Forward primer | Reverse primer |
| (SEQ ID NO:2) | (SEQ ID NO:3) |

Here the reverse primer is shown with a fluorescent label attached to its 5′ end to facilitate purification. “FAM” is a fluorescein dye available commercially, e.g. PE Applied Biosystems (Foster City, Calif.). Alternatively, the 64 double stranded oligonucleotides containing the two-word combinations may be constructed by separately synthesizing both strands and then annealing them together for cloning into a conventional cloning vector.

In embodiments where synthesis errors are eliminated by “self-selection” (described more fully below), the oligonucleotide of Formula I may be synthesized combinatorially, as disclosed in Brenner et al, International patent application PCT/US96/09513, so that a mixture of oligonucleotides is produced, the components of the mixture being oligonucleotides having different words. For example, if the four-base words of Table I are employed, then the mixture corresponding to Formula I would consist of 64 different sequences, i.e. every possible two-word sequence. In embodiments where synthesis errors are eliminated by confirmatory sequencing, the oligonucleotides of Formula I are synthesized separately followed by separate insertion into cloning vectors and sequencing to confirm that each word sequence is correct. As above, if the four-base words of Table I are employed, then 64 separate clonings and sequence determinations would be required. After such confirmatory sequencing, the 64 clones are combined for use in the method of the invention.

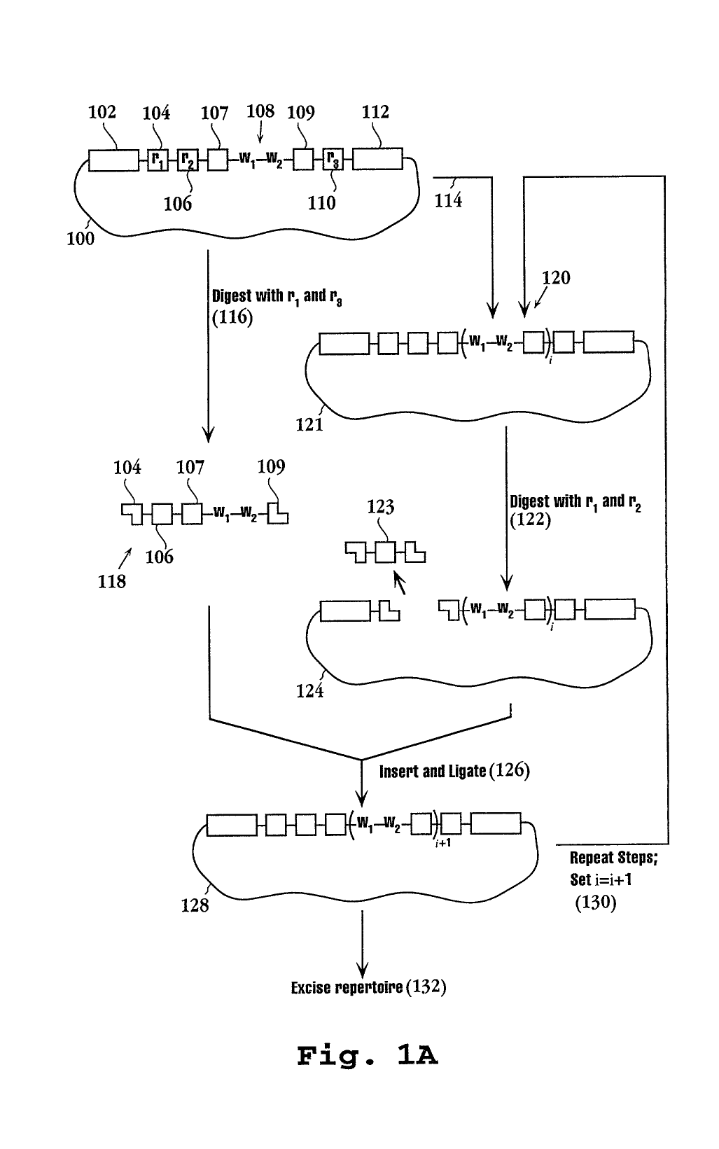

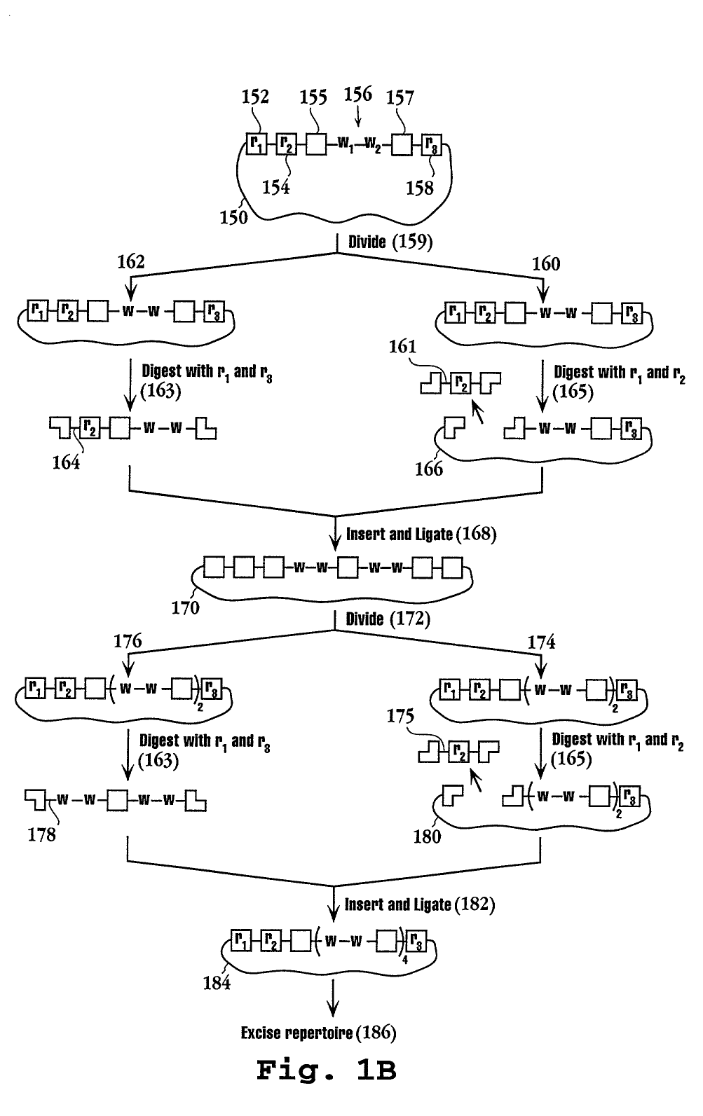

Oligonucleotide tags produced by way of the invention may be assembled from words or sub-assemblies of words either by stepwise additions in a plurality of cycles of cleavage and ligation of preferably identically sized adaptors, or in stages of convergent assembly of fragments, each of such fragments comprising increasingly larger oligonucleotide precursors. Examples of both approaches are illustrated in FIGS. 1a (stepwise additions) and 1b (convergent assembly). In FIG. 1a, vector (100) is prepared for each sequence of words “-w₁-w₂-”. The presence of two words in this example is only for purposes of illustration. In this embodiment, any number of words can be used. The practical constraint is the requirement that vector (100) be prepared for every sequence of words. Thus, if three four-base words of Table I are employed, then 512 (=8×64) vectors must be prepared and their sequences confirmed.

Adjacent to words (108) are cleavage sites (107) and (109) of type IIs restriction endonucleases, r₂ and r₃, recognizing sites (106) and (110), respectively. Adjacent to, and upstream of, restriction site (106) is restriction site (104) recognized by restriction endonuclease, r₁. Flanking the entire assembly of restriction sites and words are optional primer binding sites (102) and (112), which may be used to copy the oligonucleotide tag for insertion into a vector as taught by Brenner et al, International application pct/us96/09513.

In the preferred embodiment of FIG. 1a, vector (100) serves (114) as a starting material for the tag assembly process, i.e. at the start of the process, i=1 in the subscript of insert (120). Note that the process entails the successive insertion of the following element, or cassette:

−w−w−(N)ₖ−

where “w” is a word, “N” is a nucleotide, and k is an integer equal to 1, 2, 3, or 4. The term “(N)ₖ” is equivalent to element (109) of FIG. 1a. As described above, preferably k is equal to 1 or 2, which is the length of the protruding strand resulting from cleavage with the preferred type IIs restriction endonucleases of the invention. r₃ is virtually any type IIs restriction endonuclease which allows a predictable sequence (109) to be engineered into vector (100). Exemplary r₃′s include Alw I, Bbs I, Bbv I, Bci VI, Bpm I, Bsa MI, Bse GI, Bsr DI, Ear I, Fau I, Mbo II, and the like. Preferably, r₃ leaves a 1 or 2 nucleotide protruding strand after cleavage. Likewise, r₂ is virtually any type IIs restriction endonuclease which allows a predictable sequence (107) to be engineered into vector (100). r₂ may be selected from the same group of type IIs restriction endonucleases as r₃, but preferably for a given vector r₁ and r₂ are different.

Cycles of word addition in the preferred embodiment, illustrated in FIG. 1a, begin with the step of cleaving (122) vector (121) with r₁ and r₂, to remove segment (123), thereby leaving opened vector (124), which is then isolated using conventional protocols. In this embodiment, r₂ cleaves the oligonucleotide tag precursor at the upstream-most word of the tag. Separately, restriction endonucleases r₁ and r₃ recognizing restriction sites (104) and (110), respectively, are used to cleave (116) vector (100) to produce fragment (118),. which is inserted (126) into opened vector (124) to form vector (128), thereby elongating the oligonucleotide tag precursors by two words. The cycles are repeated (130) until an oligonucleotide tag repertoire of the desired length is obtained. At such point, the oligonucleotide tags may be excised from vector (128) by digesting with r₂ and r₃.

Alternatively, repertoires may be synthesized in accordance with the invention with a convergent strategy as illustrated in FIG. 1b. Vector (150), which may be identical to vector (100), contains the following elements: restriction site (152) for restriction endonuclease, r₁, restriction site (154) for restriction endonuclease r₂, which has cleavage site (155), one or more words (156), and restriction site (158), which has cleavage site (157). Optionally, vector (150) may also contain flanking primer binding sites as with vector (100) (not shown) for producing copies of the oligonucleotide tags or their precursors. Two aliquots (160) and (162) are taken of vector (150). In aliquot (160), vector (150) is digested with r₁ and r₂ so that fragment (161) is excised and opened vector (166) is formed. Separately, in aliquot (162), vector (150) is digested with r₁ and r₃ so that 2-word fragment (164) is excised. After purification, 2-word fragment (164) is inserted and ligated (168) into opened vector (166) to form vector (170), which contains oligonucleotide tag precursors consisting of four words each. These steps are repeated using vector (170) as the starting material. That is, two aliquots (174) and (176) are taken of vector (170). In aliquot (174), vector (170) is digested with r₁ and r₂ so that fragment (175) is excised and opened vector (180) is formed. Separately, in aliquot (176), vector (170) is digested with r₁ and r₃ so that 4-word fragment (178) is excised. After purification, 4-word fragment (178) is ligated (182) into opened vector (184) to form vector (184), which contains oligonucleotide tag precursors consisting of eight words each. Additional cycles may be carried out, or if the desired length of the tags is 8 words, then the oligonucleotide tags may be excised (186) by digesting with r₂ and r₃.

Repertoires of oligonucleotide tags may also be produced in accordance with the invention by repeated additions of words with self-selection during the ligation step. In this embodiment, the length of the protruding strand produced by cleavage with a type IIs restriction endonuclease is the same as the length of a word. When an oligonucleotide tag precursor is cleaved at a word, cleavage occurs precisely at the upstream and downstream boundaries of a word, i.e. across a word, as shown below:

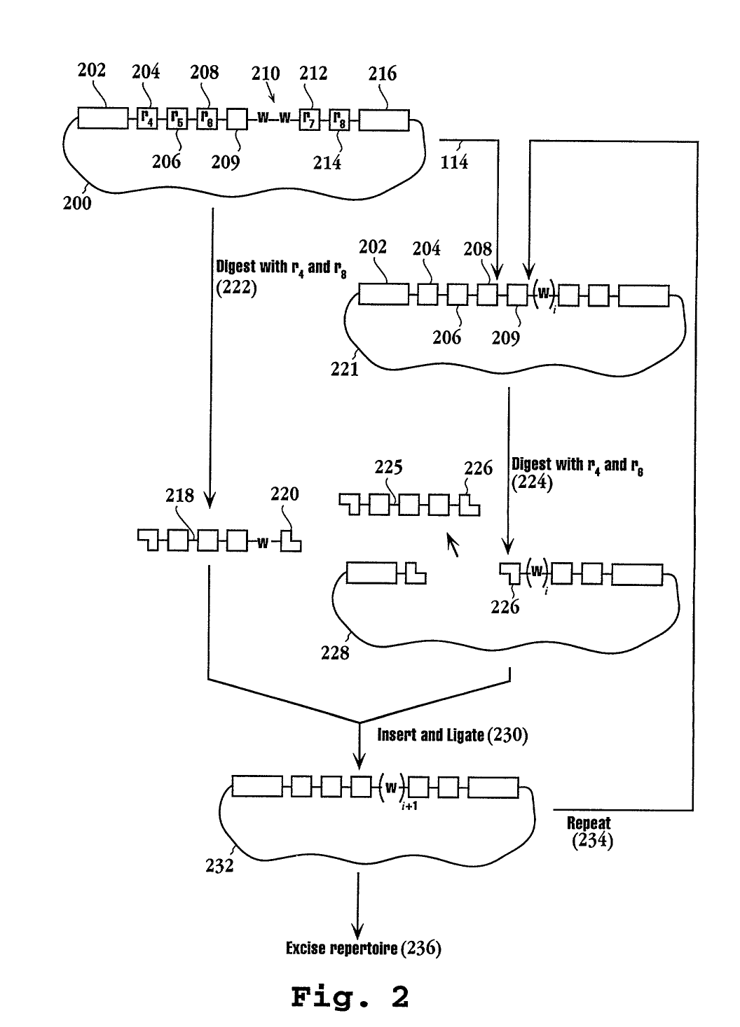

```
                      cleavage site
                              ↓
      5′-... nnnn-xxxx-xxxx-xxxx-nnnnn ...
      3′-... nnnn-xxxx-xxxx-xxxx-nnnnn ...
                                        ↑
                                cleavage site
                                            ↓
5′-... nnnn xxxx-xxxx-xxxx-nnnnn ...
3′-... nnnn-xxxx xxxx-xxxx-nnnnn ...
```

where the segments “-xxxx-” represent words consisting of four nucleotides each. Preferably, in this embodiment, word lengths of either 3, 4, or 5 nucleotides are employed. A preferred implementation of this embodiment is illustrated in FIG. 2. Vector (200), produced from conventional starting materials, includes the following elements: restriction site for r₄ (204), restriction site for r₅ (206), restriction site for r₆ (208), cleavage site (209), a plurality of words (210), restriction site for r₇ (212), and a restriction site for r₈ (214). As with vector (100), the above series of elements may be flanked by optional primer binding sites (202) and (216) so that the oligonucleotide tag precursors may be conveniently replicated, e.g. by PCR amplification.

Vector (221), which may be a sample of starting vector (200) or a previously processed vector, is cleaved (224) with r₄ and r₆ to produce fragment (225) and opened vector (228), which is isolated using conventional protocols. r₆ is a type IIs restriction endonuclease which cleave across the upstream-most word of the oligonucleotide tag precursor of vector (228). Vector (228) is actually a mixture by virtue of the different oligonucleotide tag precursors. In particular, the protruding strand of end (226) is present in N different sequences, where N is the number of words in the minimally cross-hybridizing set being used. Separately, a sample of vector (200) is cleaved (222) with r₄ and r₈ to produce fragment (218), which is isolated. Fragment (218) is a mixture containing N² components in this example, where again N is the number of words in the minimally cross-hybridizing set being used. N is to the second power because the fragment contains all possible combinations of to consecutive words. Element (220) of fragment (218) is the single-stranded form of the second, or downstream-most, word of vector (200). Fragment (218) is combined with opened vector (228) under conditions that permit the single stranded forms of the words (220) and (226) to form perfectly matched duplexes. Because of the minimally cross-hybridization property of the protruding strands, these conditions are readily met. Strands that are not complementary or that contain failure sequences will not form perfectly matched duplexes and will not be ligated. In this sense, the words in the protruding strands are “self-selecting.” After insertion and ligation (230), vector (232) is formed which contains and elongated oligonucleotide tag precursor. The cleavage and insertion steps are repeated (234) until an oligonucleotide tag of the desired length is obtained, after which the oligonucleotide tag repertoire may be excised by cleaving with r₇ and r₅.

The following examples serve to illustrate the present invention and are not meant to be limiting. Selection of many of the reagents, e.g. enzymes, vectors, and other materials; selection of reaction conditions and protocols; and material specifications, e.g. word length and composition, tag length, repertoire complexity, and the like, are matters of design choice which may be made by one of ordinary skill in the art. Extensive guidance is available in the literature for applying particular protocols for a wide variety of design choices made in accordance with the invention, e.g. Sambrook et al, Molecular Cloning, Second Edition (Cold Spring Harbor Laboratory, New York, 1989); Ausubel et al, editors, Current Protocols in Molecular Biology (John Wiley & Sons, New York, 1997); and the like.

### EXAMPLE 1

### Repertoire Synthesis by Repeated Cycles of Cleavage, Self-Selection, Ligation, and Amplification

In this example, an oligonucleotide tag repertoire is produced such that each oligonucleotide tag consists of eight words of four nucleotides. The procedure outlined in FIG. 2 is followed. A vector, corresponding to vector (200), is constructed by first inserting the following oligonucleotide (SEQ ID NO: 4) into a Bam HI and Eco RI digested pUC 19:

```
                Pac I Bse RI Bsp 120 Bbs I Eco RI Bam HI
                  ↓ ↓ ↓ ↓ ↓ ↓
aattgttaattaaggatgagctcactcctcgggcccgcataagtcttcgaattcg
        caattaattcctactcgagtgaggagcccgggcgtattcagaagcttaagcctag
                                                      Formula II
```

Separately, the oligonucleotide of Formula I and forward and reverse primers (SEQ ID NO: 2 and SEQ ID NO: 3) are synthesized using a conventional DNA synthesizer, e.g. PE Applied Biosystems (Foster City, Calif.) model 392. The oligonucleotide of Formula I is a mixture containing a repertoire of 64 two-word oligonucleotide tag precursors. The four-nucleotide words of Table I are employed. After amplification by PCR, the amplification product is digested with Bbs I to give the following two products:

| | |
|---|---|
| ... gaagacga | word-word-gg ... |
| ... cttctgct-word | word-cc ... |

The products are re-ligated, amplified by PCR, and digested with Bbv I to give the following two products:

| | |
|---|---|
| ... gaagacga-word | word-gg ... |
| ... cttctgct-word-word | cc ... |

The products are again re-ligated and amplified by PCR. By this sequence of cleavages and relations, any words consisting of failure sequences are selected against by the ligation event, i.e. words with failure sequences will not religate in the mixture, and thus, will not be amplified. The final product is digested with Pst I and Hind III and inserted into a Pst I/Hind III-digested pUC 19 to give the following construct (SEQ ID NO: 5):

```
                Pst I Bse RI Bbs I Bsp 120 Hind III
                  ↓ ↓ ↓ ↓ ↓
... cgacctgcagaggagatgaagacga-wordword-gggcccaatgctgcaagcttggcg ...
... gctggacgtctcctctacttctgct-wordword-cccgggttacgacgttcgaaccgc ...
                                                                                                        ↑
                                                                                                      Bbv I
```

where Pst I, Bse RI, Bbs I, Bsp 120, and Bbv I, correspond to r₄, r₅, r₆, r₇, and r₈ of FIG. 2, respectively. After amplification in a suitable host, the plasmid is isolated and cleaved with Pst I and Bbs I to give an opened vector with the following upstream and downstream (SEQ ID NO: 6) ends:

| | |
|---|---|
| ... cgacctgca | wordword-gggcccaatgctgcaagcttggcg ... |
| ... gctgg | word-cccgggttacgacgttcgaaccgc ... |

Separately, a portion of the amplified oligonucleotide of Formula I is digested with Pst I and Bbv I to give the following fragment (SEQ ID NO: 7)

gaggagatgaagacga-word
acgtctcctctacttctgct-wordword

This fragment is inserted into the above vector opened by digestion with Bbs I and Pst I to give the following construct (SEQ ID NO: 8)

...gcagaggagatgaagacga-wordwordword-gggcccaatgctgcaagcttggcg...
...cgtctcctctacttctgct-wordwordword-cccgggttacgacgttcgaaccgc...

which contains an oligonucleotide tag precursor of three words. The steps of cleaving, inserting, and amplification are repeated until a construct containing eight words is obtained. Preferably, at each step, reactants, e.g. vectors and/or inserts, are provided in amounts that are at least ten times the complexity of the reactant. When synthesis is complete, the eight-word construct is cleaved with Bse RI and Bsp 120 and the following fragment containing the oligonucleotide tag repertoire is isolated:

(word)₈g
ct(word)₈cccgg

The isolated fragment is then inserted into the Bse RI/Bsp 120 vector of Formula II, which vector is used to transform a suitable host. The construct is ready for inserting polynucleotides, such as cDNAs, into the Eco RI restriction site to form tag-polynucleotide conjugates in accordance with the method of Brenner et al, International patent application pct/us96/09513.

Example 2

### Repertoire Synthesis by Convergent Assembly of Error-free Oligonucleotide Tag Precursors

In this example, an oligonucleotide tag repertoire is produced following the procedure outlined in FIG. 1b. Each oligonucleotide tag consists of eight words of six nucleotides each (selected from those listed in Table I) to give the repertoire having an expected complexity of 9⁸, or about 4 3×10⁷. For each of the 9×9=81 two-word combinations, an oligonucleotide (SEQ ID NO: 9) of the following form is synthesized:

```
                Pst I Bse RI Bsp 120
                  ↓ ↓ ↓
cgacacctgcagttatcggaggagatgaagacgg[word][word]gggcccatat-
                                                                  -atccgtctgcacaagcttaccg
                                                                                    ↑ ↑
                                                                                  Bsg I Hind III
                                                        Formula III
```

The oligonucleotides of Formula III are rendered double stranded and amplified by providing forward and reverse primers and conducting a PCR, as described above for the oligonucleotide of Formula I. After amplification, the oligonucleotides are separately cleaved with Pst I and Hind III and cloned into a similarly cleaved M13mp18 and suitable hosts are transformed. Clones are selected and the oligonucleotide inserts are sequenced using conventional techniques. Such selection and sequencing continue until a vector is obtained for each of the 81 two-word combinations whose sequence is confirmed to be correct. Aliquots of the vectors are then combined in equal proportions to form an 81-component mixture, after which the vectors are cleaved with Pst I and Hind III and the word-containing fragment is isolated and cloned into a similarly cleaved pUC19 to give a construct of the following form (SEQ ID NO: 10):

```
... ctgcagttatcggaggagatgaagacgg[word][word]gggcccatat-
... gacgtcaatagcctcctctacttctgcc[word][word]cccgggtata-
                                                                  -atccgtctgcacaagcttggcg ...
                                                                  -taggcagacgtgttcgaaccgc ...
```

After cloning, the population of vectors is divided into two parts, after which the vectors in one part are cleaved with Pst I and Bsg I to give the following fragment mixture (SEQ ID NO: 11):

gttatcggaggagatgaagacgg[word][word]gg
acgtcaatagcctcctctacttctgcc[word][word]

which is isolated. The vectors in the other part are cleaved with Pst I and Bse RI and the linearized word-containing vectors are isolated. The word-containing fragments are ligated into the linearized vectors to form the following construct (SEQ ID NO: 12):

```
... ctgcagttatcggaggagatgaagacgg[word][word]gg[word][word]-
... gacgtcaatagcctcctctacttctgcc[word][word]cc[word][word]-
                                -gggcccatatatccgtctgcacaagcttggcg ...
                                -cccgggtatataggcagacgtgttcgaaccgc ...
```

After cloning, the construct is again divided into two parts and the steps are repeated to give the final 8-word repertoire having the form:

.. gaagacgg([word][word]gg)₄gccc ...
.. cttctgcc([word][word]cc)₄cggg ...

This may then be cleaved with Bse RI and Bsg I and re-cloned into a vector similar to that of Formula II for attachment to polynucleotides.

### EXAMPLE 3

### Construction of an Eight-Word Tag Library

In this example, an eight-word tag library with four-nucleotide words was constructed from two two-word libraries in vectors pLCV-2 and pUCSE-2. Prior to construction of the eight-word tag library, 64 two-word double stranded oligonucleotides were separately inserted into pUC 19 vectors and propagated. These 64 oligonucleotides consisted of every possible two-word pair made up of four-nucleotide word selected from an eight-word minimally cross-hybridizing set described in Brenner, U.S. Pat. No. 5,604,097. After the identities of the inserts were confirmed by sequencing, the inserts were then amplified by PCR and equal amounts of each amplicon were combined to form the inserts of the two-word libraries in vectors, pLCV-2 and pUCSE-2. These were then used as described below to form an eight-word tag library in pUCSE, after which the eight-word insert was transferred to vector pNCV3 which contains additional primer binding sites and restriction sites to facilitate tagging and sorting polynucleotide fragments.

A. Construction of Two-word Sequences in pUCSE pUC 19 was digested to completion with Sap I and Eco RI using the manufacturer's protocol and the large fragment was isolated. All restriction endonucleases unless otherwise noted were purchased from New England Biolabs (Beverly, Mass.). The small Sap I-Eco RI fragment was removed to eliminate the β-gal promoter sequence, which was found to skew the representation of some combinations of words in the final library. The following adaptor (SEQ ID NO: 13) was ligated to the isolated large fragment in a conventional ligation reaction to give plasmid pUCSE as a ligation product.

```
Eco RI Pst I Eco RV Hind III
    ↓ ↓ ↓ ↓
aattctagactgcagttgatatcttaagctt
        gatctgacgtcaactatagaattcgaacga
```

A bacterial host was transformed by the ligation product using electroporation, after which the transformed bacteria were plated, a clone was selected, and the insert of its plasmid was sequenced for confirmation. pUCSE isolated from the clone was then digested with Eco RI and Hind III using the manufacturer's protocol and the large fragment was isolated. The following adaptor (SEQ ID NO: 14) was ligated to the large fragment to give plasmid pUCSE-D1 which contained the first di-word (underlined).

```
                    BseRI
  EcoRI PstI BbsI Bsp120I HindIII
      ↓ ↓ ↓ ↓ ↓ ↓
aattctgcagaggagatgaagacgaaaagaaaggggcccatgctgca
        gacgtctcctctacttctgcttttctttccccgggtacgacgttcga
                                                                                          ↓
                                                                                        BbvI
                                                Formula I
```

Further plasmids, pUCSE-D2 through pUCSE-D64, containing di-words were separately constructed from pUCSE-D1 by digesting it with Pst I and Bsp 120 I and separately ligating the following adaptors (SEQ ID NO: 15) to the large fragment.

```
        gaggagatgaagacga[word][word]g
acgtctcctctacttctgct[word][word]cccgg
                                Formula II
```

The words of the top strand were selected from the following minimally cross-hybridizing set: gatt, tgat, taga, ttg, gtaa, agta, atgt, and aaag. After cloning and isolation, the inserts of the vectors were sequenced to confirm the identities of the di-words.

B. Construction pLCV

Plasmid cloning vector pLCV-D1 was created from plasmid vector pBC.SK⁻ (Stratagene) as follows, using the following oligonucleotides:

| | |
|---|---|
| S-723 | (SEQ ID NO:16) |

5′-CGA GAA AGA GGG ATA AGG CTC GAG CTT AAT TAA
GAG TCG ACG AAT TCG GGC CCG GAT CCT GAC TCT TTC
TCC CT-3′

| | |
|---|---|
| S-724 | (SEQ ID NO:17) |

5′-CTA GAG GGA GAA AGA GTC AGG ATC CGG GCC CGA
ATT CGT GGA CTC TTA ATT AAG CTC GAG CCT TAT CCC
TCT TTC TCG GTA C-3′

| | |
|---|---|
| S-785 | (SEQ ID NO:18) |

5′-TCG AGG CAT AAG TCT TCG AAT TCC ATC ACA CTG GGA
AGA CAA CGT AG-3′

| | |
|---|---|
| S-786 | (SEQ ID NO:19) |

5′-GAT CCT ACG TTG TCT TCC CAG TGT GAT GGA ATT CGA
AGA CTT ATG CC-3′

| | |
|---|---|
| S-960 | (SEQ ID NO:20) |

5′-TCG ATT AAT TAA CAA GCT TTG GGC CCT CGA GCA
TAA GTC TTC TGC AGA ATT CGG ATC CAT CGA TGG TCA
TAG C-3′

| | |
|---|---|
| S-961 | (SEQ ID NO:21) |

5′-TGT TTC CTG CCA CAC AAC ATA CGA GCC GGA AGC GGC
CGC TCT AGA-3′

| | |
|---|---|
| S-962 | (SEQ ID NO:22) |

5′-AGC GTC TAG AGC GGC CGC TTC CGG CTC GTA TGT TGT
GTG GCA GGA AAC AGC TAT GAC CAT C-3′

| | |
|---|---|
| S-963 | (SEQ ID NO:23) |

5′-GAT GGA TCC GAA TTC TGC AGA AGA CTT ATG CTC GAG
GGC CCA AAG CTT GTT AAT TAA-3′

| | |
|---|---|
| S-1105 | (SEQ ID NO:24) |

5′-TCGA GGG CCC GCA TAA GTC TTC-3′

| | |
|---|---|
| S-1106 | (SEQ ID NO:25) |

5′-TCGA GAA GAC TTA TGC GGG CCC-3′

Oligonucleotides S-723 and S-724 were kinased, annealed together, and ligated to pBC.SK⁻ which had been digested with KprI and XbaI and treated with calf intestinal alkaline phosphatase, to create plasmid pSW143.1.

Oligonucleotidess S-785 and S-786 were kinased, annealed together, and ligated to plasmid pSW143.1, which had been digested with XhoI and BamHI and treated with calf intestinal alkaline phosphatase, to create plasmid pSW164.02.

Oligonucleotides S-960, S-961, S-962, and S-963 were kinased and annealed together to form a duplex consisting of the four oligonucleotides. Plasmid pSW164.02 was digested with XhoI and SapI. The digested DNA was electrophoresed in an agarose gel, and the approximately 3045 bp product was purified from the approprate gel slice. Plasmid pUC4K (from Pharmacia) was digested with PstI and electrophoresed in an agarose gel. The approx. 1240 bp product was purified from the appropriate gel slice. The two plasmid products (from pSW164.02 and pUC4K) were ligated together with the S-960/961/962/963 duplex to create plasmid pLCVa.

DNA from Adenovirus5 (New England Biolabs) was digested with PacI and Bsp120I, treated with calf intestinal alkaline phosphatase, and electrophoresed in an agarose gel. The approx. 2853 bp product was purified from the appropriate gel slice. This fragment was ligated to plasmid pLCVa which had been digested with PacI and Bsp120I, to create plasmid pSW208.14.

Plasmid pSW208.14 was digested with XhoI, treated with calf intestinal alkaline phosphatase, and electrophoresed in an agarose gel. The approx. 5374 bp product was purified from the appropriate gel slice. This fragment was ligated to oligonucleotides S-1105 and S-1106 (which had been kinased and annealed together) to produce plasmid pLCVb, which was then digested with Eco RI and Hind III. The large fragment was isolated and ligated to the Formula I adaptor (SEQ ID NO: 14) to give pLCV-D1.

As above for pUCSE, further plasmids, pLCV-D2 through pLCV-D64, containing di-words were separately constructed from pLCV-D1 by digesting it with Pst I and Bsp 120 I, isolating the large fragment, and a ligating an adaptor of Formula II. After cloning and isolation, the inserts of the vectors were sequenced to confirm the identities of the di-words

C. Construction of Two-word Libraries, pUCSE-2 and pLCV-2.

Each of the vectors pLCV-D1 through -D64 and pUCSE-D1 through -D64 was separately amplified by PCR. The components of the reaction mixture were as follows:

| | | |
|---|---|---|
| 10 | μl | template (about 1-5 ng) |
| 10 | μl | 10x Klentaq ™ buffer (Clontech Laboratories, Palo Alto, CA) |
| 2.5 | μl | biotinylated DF primer at 100 pmoles/μl |
| 2.5 | μl | biotinylated DR primer at 100 pmoles/μl |
| 2.5 | μl | 10 mM deoxynucleoside triphosphates |
| 5 | μl | DMSO |
| 66.5 | μl | H₂O |
| 1 | μl | Advantage Klentaq ™ (Clontech Laboratories, Palo Alto, CA) |

The temperature of the reactions was controlled as follows: 94° C. for 3 min; 25 cycles of 94° C. for 30 sec, 60° C. for 30 sec, and 72° C. for 10 sec; followed by 72° C. for 3 min., then 4° C. The DF and DR primer binding sites were upstream and downstream portions of the vectors selected to give amplicons of 104 basepairs in length. After the reactions were completed, 5 μl of each PCR product were separated polyacrylamide gel electrophoresis (20% with 1×TBE) to confirm by visual inspection that the reaction yields were approximately the same for each PCR. After such confirmation, using conventional protocols, 10 μl of each PCR was extracted twice with phenol and once with chloroform, after which the DNA in the aqueous phase was precipitate with ethanol.

After resuspension in 200 μl of 1×NEB buffer #2 (New England Biolabs, Beverly, Mass.), the DNA was cleaved with Bbv I and Eco RI by adding the enzymes in 50 μl of the manufacturer's recommended buffer. The digestion resulted in the production of three fragments: a biotinylated fragment of 38 basepairs, a di-word-containing fragment of 29 basepairs, and a biotinylated fragment of 37 basepairs. After completion of the reaction, the excess biotinylated primers were removed by adding 50 μl 50% Ultralink (streptavidin-Sepharose, Pierce Chemical Co., Rockford, Ill.) and vortexing the mixture at room temperature for 30 min. The Ultralink material was separated from the reaction mixture by centrifugation, after which approximately half of the mixture was separated by polyacrylamide gel electrophoresis (20% gel). The 29-basepair band was cut out of the gel and the 29-basepair fragment was eluted using the “crush and soak” method, e.g. Sambrook et al, Molecular Cloning, Second Edition (Cold Spring Harbor Laboratory, New York, 1989). This material was then ligated into either pLCV-D1 or pUCSE-D1 after the latter were digested with Bbs I and Eco RI and treated with calf intestine alkaline phosphatase, using manufacturer's recommend protocols.

D. Construction of pNCV3.

pNCV3 was constructed by first assembling the following fragment (SEQ ID NO: 26) from synthetic oligonucleotides:

```
EcoRI
    ↓
aattctgtaaaacgacggccagtcgccagggttttcccagtcacgacgtgaataaatag-
        gacattttgctgccggtcagcggtcccaaaagggtcagtgctgcacttatttatc-
      PacI Bsp120I
        ↓ ↓
ttaattaaggaataggcctctcctcgagctcggtaccgggcccgcataagtcttc-
aattaattccttatccggagaggagctcgagccatggcccgggcgtattcagaag-
            ClaI EcoRV SapI BamHI
              ↓ ↓ ↓ ↓
atctatcgatgattgaagagcgatatcgctcttcaatcggatccatcc-
tagatagctactaacttctcgctatagcgagaagttagcctaggtagg-
                                    ↑
                                SapI
                                                                                                  HindIII
                                                                                                      ↓
tcaactaattaccacacaacatacgagccggaagcgggtcatagctgtttcctga
agttgattaatggtgtgttgtatgctcggccttcgcccagtatcgacaaaggacttcga
```

After isolation, the fragment was cloned into Eco RI and Hind III-digested pLCV-D1 using conventional protocols.

E. Assembly of Eight-word Library.

The di-words of pLCV-2 were amplified either by PCR or plasmid expansion, the product was digested with Eco RI and BbvI after which the Eco RI-BbvI fragment was isolated as insert 1. Two-word library pUCSE-2 was digested with Eco RI, Bbs I, and Pst I, after which the large fragment was treated with calf intestine alkaline phosphatase to give vector 1. Vector 1 and insert 1 were combined in a conventional ligation reaction to give three-word library, pUCSE-3. pUCSE-3 was digested with Eco RI, Bbs I., and Pst I, after which the large fragment was treated with calf intestine alkaline phosphatase to give vector 2. Vector 2 and insert 1 were then combined in a conventional ligation reaction to give four-word library, pUCSE-4. The 4-mer words of pUCSE-4 were amplified either by PCR or plasmid expansion, the product was digested with Eco RI and BbvI after which the Eco RI-BbvI fragment was isolated as insert 2. pLCV-2 was digested with Eco RI, Bbs I, and Pst I, after which the large fragment was treated with calf intestine alkaline phosphatase to give vector 3. Vector 3 and insert 2 were then combined in a conventional ligation reaction to give five-word library, pLCV-5. The 5-mer words of pLCV-5 were amplified either by PCR or plasmid expansion, the product was digested with Eco RI and BbvI after which the Eco RI-BbvI fragment was isolated as insert 3. pUCSE-4 was digested with Eco RI, Bbs I, and Pst I, after which the large fragment was treated with calf intestine alkaline phosphatase to give vector 4. Vector 4 and insert 3 were then combined in a conventional ligation reaction to give eight-word library, pUCSE-8. The 8-mer words of pUCSE-8 were amplified either by PCR or plasmid expansion, the product was digested with Bse RI and Bsp120 I, after which the BseRI-Bsp 120I fragment was isolated as insert 4. pNCV3 was digested with Bse RI, Bsp120 I, and Sac I, after which the large fragment was isolated and treated with calf intestine alkaline phosphatase to give vector 5. Vector 5 was then combined with insert 4 in a conventional ligation reaction to give the eight-word library pNCV3-8.

F. Confirmation Sequencing of a Random Selection of Eight-Word Tags

The results of the word assembly were tested by sequencing the 8-word inserts of 176 vectors from the pNCV3-8 library. The results of the sequence determinations are summarized in the following table:

| Number of Tags | Result | Percentage |
|---|---|---|
| 147 | Perfect 8 words | 83.5% |
| 11 | Perfect 7 words | 6.2% |
| 8 | No insert | 4.5% |
| 4 | 8 words with 1 base deletion | 2.2% |
| 3 | 8 words with an incorrect word | 1.7% |
| 1 | 12 words | 0.5% |
| 1 | 10 words | 0.5% |
| 1 | 9 words | 0.5% |

We claim:

1. A method of synthesizing a repertoire of oligonucleotide tags of a predetermined length, the method comprising the steps of:
(a) providing a repertoire of oligonucleotide tag precursors in an amplicon, the oligonucleotide tag precursors each comprising one or more words, and each of the one or more words being selected from the same minimally cross-hybridizing set;
(b) cleaving the amplicon at a word in each of the oligonucleotide tag precursors to form one or more ligatable ends on each oligonucleotide tag precursor;
(c) ligating one or more words to the one or more ligatable ends to elongate each of the oligonucleotide tag precursors;
(d) amplifying the elongated oligonucleotide tag precursors in the amplicon; and
(e) repeating steps (b) through (d) until a repertoire of oligonucleotide tags having the predetermined length is formed.

2. The method of claim 1 wherein said amplicon is a cloning vector.

3. The method of claim 2 wherein said step of cleaving includes cleaving said amplicon in a region adjacent to said word by a type IIs restriction endonuclease.

4. The method of claim 3 wherein said word has a length in the range of from three to fourteen nucleotides.

5. The method of claim 4 wherein oligonucleotide tag has a length in the range of from 18 to 60 nucleotides.

6. The method of claim 2 wherein said step of cleaving includes cleaving said amplicon across said word by a type IIs restriction endonuclease.

7. The method of claim 2 wherein said word has a length of four and wherein said oligonucleotide tag has a length in the range of from 18 to 40.

8. A repertoire of oligonucleotide tags, wherein the oligonucleotide tags of the repertoire are of the form:
w₁(N)ₓ₁w₂(N)ₓ₂ . . . (N)ₓₙ₋₁wₙ
wherein each of w₁ through wₙ is a word consisting of an oligonucleotide having a length from three to fourteen nucleotides or basepairs and being selected from the same minimally cross-hybridizing set wherein a word of the set and a complement of any other word of the set has at least two mismatches; N is a nucleotide or basepair; each of x₁ through xₙ₋₁ is an integer selected from the group consisting of 0, 1, 2, 3, and 4, provided that at least one of x₁ through xₙ₋₁ is 1, 2, 3, or 4; and n is an integer in the range of from 4 to 10.

9. The repertoire of claim 8 wherein each of said x₁ through xₙ₋₁ is selected from the group consisting of 0, 1, and 2, and wherein said length of said word is from four to ten nucleotides or basepairs.

10. The repertoire of claim 9 wherein said oligonucleotide tags are single stranded and wherein n is in the range of from 6 to 10.

11. The repertoire of claim 10 wherein a duplex between each of said words of said minimally cross-hybridizing set and said complement of any other word of said set would have at least three mismatches.

12. The repertoire of claim 11 wherein a duplex between each of said words of said minimally cross-hybridizing set and said complement of any other word of said set would have at least five mismatches whenever said word has a length of greater than or equal to six nucleotides.

13. The repertoire of claim 10 having a number of said oligonucleotide tags that is in the range of from 100 to 1×10⁹.

14. The repertoire of claim 13 having a number of said oligonucleotide tags that is in the range of from 1000 to 1×10⁸.

15. A repertoire of cloning vectors for attaching oligonucleotide tags to polynucleotides, wherein each of the vectors comprises a double stranded element corresponding to an oligonucleotide tag of the form:
w₁(N)ₓ₁w₂(N)ₓ₂ . . . (N)ₓₙ₋₁wₙ
wherein each of w₁ through wₙ is a word consisting of an oligonucleotide having a length from three to fourteen nucleotides and being selected from the same minimally cross-hybridizing set wherein a word of the set and a complement of any other word of the set has at least two mismatches; N is a nucleotide; each of x₁ through xₙ₋₁ is an integer selected from the group consisting of 0, 1, 2, 3, and 4, provided that at least one of x₁ through xₙ₋₁ is 1, 2, 3, or 4; and n is an integer in the range of from 4 to 10.

16. The repertoire of claim 15 wherein each of said x₁ through xₙ₋₁ is selected from the group consisting of 0, 1, and 2, and wherein said length of said word is from four to ten nucleotides or basepairs.


SEQUENCE LISTING (37 sequences)

SEQ ID NO: 1 - DNA, 58 bp, Artificial Sequence
  synthetic oligonucleotide
cgacacctgcagaggagatgaagacgaddddddddgggcccatgctgcaagcttaccg

SEQ ID NO: 2 - DNA, 17 bp, Artificial Sequence
  primer
cgacacctgcagaggag

SEQ ID NO: 3 - DNA, 17 bp, Artificial Sequence
  primer
cggtaagcttgcagcat

SEQ ID NO: 4 - DNA, 55 bp, Artificial Sequence
  adaptor
aattgttaattaaggatgagctcactcctcgggcccgcataagtcttcgaattcg

SEQ ID NO: 5 - DNA, 57 bp, Artificial Sequence
  cloning vector
cgacctgcagaggagatgaagacgaddddddddgggcccaatgctgcaagcttggcg

SEQ ID NO: 6 - DNA, 32 bp, Artificial Sequence
  vector
ddddddddgggcccaatgctgcaagcttggcg

SEQ ID NO: 7 - DNA, 20 bp, Artificial Sequence
  adaptor
gaggagatgaagacgadddd

SEQ ID NO: 8 - DNA, 55 bp, Artificial Sequence
  vector
gcagaggagatgaagacgaddddddddddddgggcccaatgctgcaagcttggcg

SEQ ID NO: 9 - DNA, 78 bp, Artificial Sequence
  tag repertoire
cgacacctgcagttatcggaggagatgaagacggddddddddddddgggcccatatatccgtctgcacaagcttaccg

SEQ ID NO: 10 - DNA, 72 bp, Artificial Sequence
  vector
ctgcagttatcggaggagatgaagacggddddddddddddgggcccatatatccgtctgcacaagcttaccg

SEQ ID NO: 11 - DNA, 37 bp, Artificial Sequence
  adaptor
gttatcggaggagatgaagacggddddddddddddgg

SEQ ID NO: 12 - DNA, 86 bp, Artificial Sequence
  vector
ctgcagttatcggaggagatgaagacggddddddddddddggddddddddddddgggcccatatatccgtctgcacaagcttaccg

SEQ ID NO: 13 - DNA, 31 bp, Artificial Sequence
  adaptor
aattctagactgcagttgatatcttaagctt

SEQ ID NO: 14 - DNA, 47 bp, Artificial Sequence
  adaptor
aattctgcagaggagatgaagacgaaaagaaaggggcccatgctgca

SEQ ID NO: 15 - DNA, 25 bp, Artificial Sequence
  adaptor
gaggagatgaagacgaddddddddg

SEQ ID NO: 16 - DNA, 74 bp, Artificial Sequence
  synthetic oligonucleotide
cgagaaagagggataaggctcgagcttaattaagagtcgacgaattcgggcccggatcctgactctttctccct

SEQ ID NO: 17 - DNA, 82 bp, Artificial Sequence
  synthetic oligonucleotide
ctagagggagaaagagtcaggatccgggcccgaattcgtcgactcttaattaagctcgagccttatccctctttctcggtac

SEQ ID NO: 18 - DNA, 47 bp, Artificial Sequence
  synthetic oligonucleotide
tcgaggcataagtcttcgaattccatcacactgggaagacaacgtag

SEQ ID NO: 19 - DNA, 47 bp, Artificial Sequence
  vector
gatcctacgttgtcttcccagtgtgatggaattcgaagacttatgcc

SEQ ID NO: 20 - DNA, 72 bp, Artificial Sequence
  synthetic oligonucleotide
tcgattaattaacaagctttgggccctcgagcataagtcttctgcagaattcggatccatcgatggtcatag

SEQ ID NO: 21 - DNA, 45 bp, Artificial Sequence
  synthetic oligonucleotide
tgtttcctgccacacaacatacgagccggaagcggccgctctaga

SEQ ID NO: 22 - DNA, 62 bp, Artificial Sequence
  synthetic oligonucleotide
agcgtctagagcggccgcttccggctcgtatgttgtgtggcaggaaacaagctatgaccatc

SEQ ID NO: 23 - DNA, 57 bp, Artificial Sequence
  synthetic oligonucleotide
gatggatccgaattctgcagaagacttatgctcgagggcccaaagcttgttaattaa

SEQ ID NO: 24 - DNA, 22 bp, Artificial Sequence
  synthetic oligonucleotide
tcgagggcccgcataagtcttc

SEQ ID NO: 25 - DNA, 22 bp, Artificial Sequence
  vector
tcgagaagacttatgcgggccc

SEQ ID NO: 26 - DNA, 217 bp, Artificial Sequence
  adaptor
aattctgtaaaacgacggccagtcgccagggttttcccagtcacgacgtgaataaatagttaattaaggaataggcctctcctcgagctcggtaccgggcccgcataagtcttcatctatcgatgattgaagagcgatatcgctcttcaatcggatccatcctcaactaattaccacacaacatacgagccggaagcgggtcatagctgtttcctga

SEQ ID NO: 27 - DNA, 55 bp, Artificial Sequence
  complementary sequence to adaptor
gatccgaattcgaagacttatgcgggcccgaggagtgagctcatccttaattaac

SEQ ID NO: 28 - DNA, 10 bp, Artificial Sequence
  product of synthetic oligonucleotide digested with Bbs I
ddddddddgg

SEQ ID NO: 29 - DNA, 12 bp, Artificial Sequence
  complement to product of synthetic oligonucleotide digested with Bbs I
ddddtcgtcttc

SEQ ID NO: 30 - DNA, 12 bp, Artificial Sequence
  product of digested synthetic oligonucleotide
gaagacgadddd

SEQ ID NO: 31 - DNA, 16 bp, Artificial Sequence
  complement to product of digested synthetic oligonucleotide
ddddddddtcgtcttc

SEQ ID NO: 32 - DNA, 28 bp, Artificial Sequence
  complement to vector
cgccaagcttgcagcattgggcccdddd

SEQ ID NO: 33 - DNA, 39 bp, Artificial Sequence
  fragment containing oligonucleotide tag repertoire complement
ggcccddddddddddddddddddddddddddddddddtc

SEQ ID NO: 34 - DNA, 35 bp, Artificial Sequence
  adaptor complement
ddddddddccgtcttcatctcctccgataactgca

SEQ ID NO: 35 - DNA, 52 bp, Artificial Sequence
  eight word repertoire
gaagacggddddddddggddddddddggddddddddggddddddddgggccc

SEQ ID NO: 36 - DNA, 30 bp, Artificial Sequence
  adaptor complement
agcaagcttaagatatcaactgcggtctag

SEQ ID NO: 37 - DNA, 47 bp, Artificial Sequence
  adaptor complement
agcttgcagcatgggcccctttcttttcgtcttcatctcctctgcag